(19) United States
(12) Patent Application Publication
(10) Pub. No.: US 2005/0043532 A1
(43) Pub. Date: Feb. 24, 2005

(54) Direct synthesis of di-heteroatom containing cyclic organic compounds

(76) Inventors: Michael Siskin, Randolph, NJ (US); Edmund J. Mozeleski, Califon, NJ (US)

Correspondence Address: EXXONMOBIL RESEARCH AND ENGINEERING COMPANY, P.O. Box 900, Annandale, NJ, 08801-0900, US

(21) Appl. No.: 10/894,804

(22) Filed: Jul. 20, 2004

Related U.S. Application Data
(60) Provisional application No. 60/497,233, filed on Aug. 22, 2003.

(51) Int. Cl. C07D 279/12; C07D 265/30; C07D 339/02

(52) U.S. Cl. 544/59; 544/106; 549/20

(57) ABSTRACT

The present invention provides for the simplified, direct synthesis of di-heteroatom containing cyclic organic compounds by forming an aqueous mixture of material of the formula 1 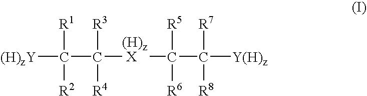
wherein X and Y are heteroatoms which may be the same or different and X is selected from the group consisting of O, S and N, and both Y's are the same and are selected from the group consisting of O, S and N, and R¹ through R⁸ are the same or different and are selected from the group consisting of H, C₁ to C₁₀ alkyl radicals, C₁ to C₁₀ aryl radicals, C₁ to C₁₀ alkyl aryl radicals, and z is zero when X and Y are O or S, and z is 1 when X and Y are N, and water at a mole ratio of water to material of formula I in the range of about 10:1 to about 0.001:1 and heating the mixture to a temperature in the range of about 250° C. to about 350° C. under autogeneous pressure for a time of from about 0.5 to 10 hours. Trace amounts of liquid, solid or gaseous acid can be present to further accelerate dehydration, dehydrosulfurization or deamination and cyclization.

### CROSS REFERENCE TO RELATED APPLICATIONS

This application claims priority from Application Ser. No. 60/497,233, filed Aug. 22, 2003, entitled “Direct Synthesis of Di-Heteroatom Containing Cyclic Organic Compounds” of M. Siskin and E. J. Mozeleski, the disclosure of which is incorporated by reference.

### FIELD OF THE INVENTION

The present invention relates to a process for the production of di-heteroatom containing cyclic organic compounds, including dioxane, oxathiane, dithiane, morpholine, piperazine or thiamorpholine.

### BACKGROUND OF THE INVENTION

Numerous processes are described in the literature for the conversion of ethers into more valuable products such as conversion into alcohols and olefins.

U.S. Pat. No. 2,045,785 teaches the conversion of ethers into alcohols by a process wherein the ether is converted using a dilute aqueous solution of an acid catalyst by subjecting the mixture to temperatures within the range of about 200° C. to 300° C. (392° F. to 572° F.) and pressure of from about 225 to 3000 psig for a period of from about 10 to 60 minutes. Acid concentration in the aqueous acid solution is in the range of about 5 to 20%. While strong mineral acids such as sulfuric, phosphoric or hydrochloric are preferred, weaker acids such as acetic are also disclosed.

U.S. Pat. No. 2,519,061 teaches a process of the cleavage of ethers into alcohols by passing an aqueous mixture of the ether over an hydration catalyst comprising metal oxides at temperatures in the range of 350° F. (177° C.) to 800° F. (427° C.) and pressures of from atmospheric up to a preferred pressure of 200 atmospheres.

U.S. Pat. No. 4,357,147 while directed to a process for the production of oxygenated fuel stock teaches that the isopropyl ether by-product is converted into propylene, isopropyl alcohol and water in a conversion step using alumina or a zeolite as a catalyst. This is related to U.S. Pat. No. 4,405,822 which teaches a process for producing alcohols by olefin hydration wherein any ether by-product is converted using a large molar excess of water in the presence of an acid ion exchange resin.

U.S. Pat. No. 4,751,343 discloses the production of tertiary-olefins. The process simultaneously produces alcohols by the cleavage of tertiary alkyl ethers when mixed with water over strong acid cation exchangers. U.S. Pat. No. 4,804,703 describes cross-linked acidic resins suitable for use in cleaving tertiary ethers. EP 302-336 teaches tertiary ether cleavage using a column apparatus containing an acidic ion exchange resin and employing a stream of deionized water.

U.S. Pat. No. 4,581,475 teaches the direct conversion of aliphatic ethers into aliphatic alcohols by reacting the ether with an excess of water at high temperature and at a pressure sufficient to keep the reactants in the liquid phase in the presence of a strong acid ion exchanger.

U.S. Pat. No. 5,043,486 teaches a simplified process of converting ethers into their corresponding alcohols comprising forming an aqueous mixture of the ether with at least about 50% by weight water and heating the mixture under autogeneous pressure at a temperature of from about 250° C. to 450° C. (482° F. to 842° F.) for a time sufficient to convert at least about 20% by weight of the ether into alcohol. The reaction proceeds in water primarily through ionic routes rather than through free radical routes to obtain relatively high conversion rates and good yields of alcohol and other reaction by-products without the necessity of using a catalyst. While water alone may be used as the aqueous medium, the use of small quantities of acid, usually less than 3% by weight may also be used in this ether-to-alcohol conversion process.

U.S. Pat. No. 5,403,964 is similarly directed to the conversion of ethers by cleavage into alcohols in the presence of water containing an anionic or cationic surfactant, preferably at least about 10⁻⁵ molar surfactant, the reaction being conducted at from about 200° C. to about 450° C. (392° F. to 842° F.).

In all of these processes, the ether is being converted into its corresponding alcohol. The resulting alcohol can be further converted into the corresponding olefin employing longer reaction times.

p-Dioxane is a valuable solvent, wetting and dispersing agent, and commercial starting material useful in numerous reactions that is manufactured by the treatment of ethylene glycol with acid or by the treatment of beta, beta-dichloroethyl ether with alkali. Both processes involve the practice of recovery steps and the separation of the p-dioxane from a reaction mixture containing either acid or alkali and various by-products.

In the production of dioxane as presented above using ethylene glycol the hydroxyl groups of two ethylene glycol molecules are involved in a dehydration reaction resulting in the production of the dioxane as outlined below:

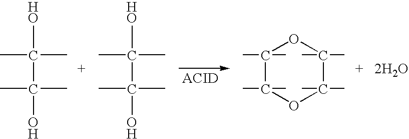

This is to be compared with the processes described in the cited patents including U.S. Pat. No. 5,043,486 and U.S. Pat. No. 5,403,964 wherein the ether is cleaved to produce alcohol using water under autogeneous pressure at 250° C. to about 450° C. (482° F. to 842° F.) and optionally in the presence of trace acid.

### SUMMARY OF THE INVENTION

The present invention is directed to the direct synthesis of di-heteroatom containing cyclic organic compounds, including p-dioxane, oxathiane, dithiane, morpholine, piperazine or thiamorpholine from 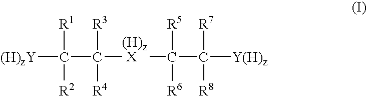
wherein X and Y are heteroatoms which may be the same or different and wherein X is selected from the group of heteroatoms consisting of O, S and N, preferably O, and both Y's are the same and selected from the group consisting of O, S and N, preferably O, and R¹, R², R³, R⁴, R⁵, R⁶, R⁷ and R⁸ are the same or different and are selected from the group consisting of hydrogen, C₁ to C₁₀ alkyl radicals, C₁-C₁₀ aryl radicals, C₁-C₁₀ aralkyl or alkyl aryl radicals, preferably hydrogen and C₁-C₄ alkyl radicals, more preferably hydrogen and methyl radicals, most preferably hydrogen, and z is zero when X and Y are O or S and z is 1 when X and Y are N, by the process of forming an aqueous mixture of the material of formula I and water at a mole ratio of water to material of formula I of between about 10:1 to about 0.001:1, preferably about 5:1 to about 0.001:1, more preferably about 1:1 to about 0.0002:1 and heating the mixture under autogeneous pressure at between about 200° C. to about 450° C. (392° F. to 842° F.), preferably about 250° C. to up to the critical temperature of water which is about 374° C. (705° F.), more preferably about 250° C to about 300° C (482° F. to 572° F.) for a time sufficient to cause the dehydration, dehydrosulfurization or deamination and ring closure of the material of formula I into a di-heteroatom containing cyclic organic compounds, including p-dioxane, oxathiane, dithiane, morpholine, piperazine or thiamorpholine. A trace amount of a solid, liquid or gaseous acid such as phosphoric acid, MCM22, acetic acid, sulfuric acid, hydrochloric acid and the like can be present to facilitate the protonation of the alcohol oxygen, the sulfhydryl sulfur or the aminic nitrogen in the material of formula I and induce dehydration, desulfurization or deamination, as the case may be, and ring closure. In the present application and in the appended claims, the term dehydration means the loss of H₂O, dehydrosulfurization means the loss of H₂S, and deamination means the loss of NH₃.

### DESCRIPTION OF THE INVENTION

It has been discovered that di-heteroatom containing cyclic organic compounds including p-dioxane, oxathiane, dithiane, morpholine, piperazine or thiamorpholine can be synthesized in a simplified process by forming an aqueous mixture of a material of formula I and water at a water to material of formula I mole ratio of about 10:1 to about 0.001:1, preferably about 5:1 to about 0.001:1 more preferably about 1:1 to about 0.002:1 and heating the mixture at between about 200° C. to about 450° C., preferably about 250° C. to up to the critical temperature of water, more preferably about 250° C. to about 300° C., at autogeneous pressure for a time sufficient to cause the dehydration, dehydrosulfurization or deamination and ring closure of the material of formula I to dioxane, oxathiane, dithiane, morpholine, piperazine or thiamorpholine, as the case may be. By di-heteroatom containing is meant that the cyclic organic compounds contain two heteroatoms in the ring which may be the same or different and are selected from the group consisting of sulfur, nitrogen and oxygen.

Water at high temperature and pressure manifests a change in its chemical and physical properties, changing from polar to almost non polar in nature; the density also decreases; it becomes miscible with organic compounds and with gases; its acidity increases. These changes are manifest most obviously and to the greatest extent below the critical point for water, a temperature of 374° C. and 218 atm, and even at near supercritical conditions water has a higher acidity than one would attribute merely to elevated temperature. It is believed the acidity (-log K_{w}@, 150° C.=11.64; @ 250° C.=11.20) is essentially three orders of magnitude greater than at 25° C. (-log K_{w}@ 25° C.=13.99) and is responsible for the catalytic activity of water.

It is surprising in the case of diethylene glycol that it would be converted into p-dioxane in near critical or super critical water because, in view of U.S. Pat. No. 5,043,486 and U.S. Pat. No. 5,403,964, one more likely would have expected the ether linkage of the diethylene glycol to have been cleaved yielding a mixture of alcohols and olefins rather than for the diethylene glycol to have been dehydrated and cyclized into p-dioxane.

This invention can utilize materials of the general formula. 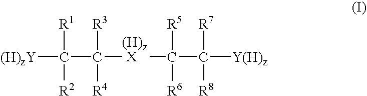
wherein X and Y are heteroatoms which may be the same or different and wherein X is selected from the group consisting of O, S and N, preferably O, and both Y's are the same and selected from the group consisting of O, S and N, preferably O, and R¹, R², R³, R⁴, R⁵, R⁶, R⁷ and R⁸ are the same or different and are selected from the group consisting of hydrogen, C₁ to C₁₀ alkyl radicals, C₁-C₁₀ aryl radicals, C₁-C₁₀ aralkyl or alkyl aryl radicals, preferably hydrogen and C₁-C₄ alkyl radicals, more preferably hydrogen and methyl radicals, most preferably hydrogen, and z is zero when X and Y are O or S, and z is 1 when X and Y are N, to yield di-heteroatom containing cyclic organic compounds, including those of the formula.

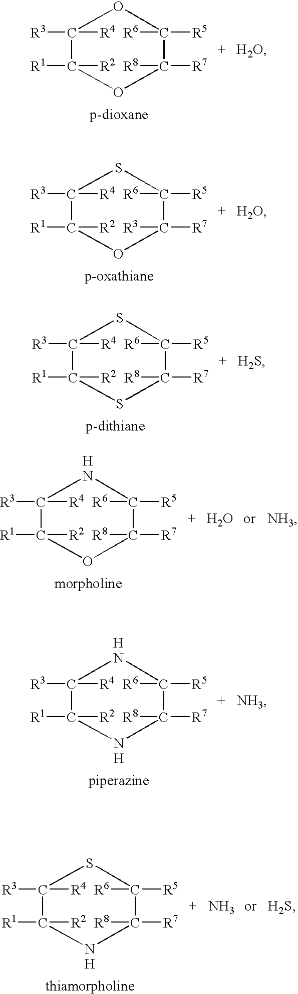

Preferably the starting material is diethylene glycol 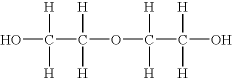
and the final product is p-dioxane

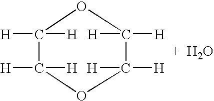

Ordinary tap water can be used in the process, but it is preferred to use distilled water, deionized water, or water that has been both distilled and deionized. Generally, the water is substantially free of dissolved salts and particularly the use of water which has been deoxygenated and is substantially free of dissolved oxygen as well as of any minerals or other ions so as to minimize any undesirable side reactions and to simplify subsequent product purification.

The amount of water used can range on a mole basis of water to material of formula I of from about 10:1 to about 0.001:1, preferably about 5:1 to about 0.001:1, more preferably about 1:1 to about 0.002:1. A ratio of<0.1 is most preferred to facilitate recovery and further the reaction to product formation. The water needed in the reaction can be that which is absorbed by the reactant or the acid catalyst from the atmosphere provided the minimum amount of water on a mole basis of about 0.001:1 H₂O to material of formula I is met.

The process is conducted by introducing the water and the material of formula I into a reaction vessel and heating the mixture under autogeneous pressure and preferably in an inert atmosphere to minimize any side reaction, the inert atmospheres including argon and/or nitrogen, the heating being at a temperature within the range of from about 200° C. to about 450° C., preferably about 250° C. to the critical temperature of water (374° C.), more preferably about 250° C. to 300° C. Heating is for a time sufficient to cause dehydration, dehydrosulfurization or deamination and ring closure of the material of formula I. Conversion time of from about 0.5 to 10 hours, preferably 1 to 5 hours, more preferably about 3-5 hours are useful.

The term “autogeneous pressure” refers to the combined vapor pressure exerted by the mixed components present in the aqueous system when heated at a particular process temperature in a closed reactor vessel. The autogeneous pressure for water alone in such a system ranges from about 500 to about 3200 psia over a temperature range of from about 250° C. up to the critical temperature of water, about 374° C. The autogeneous pressure of a system containing both water and material of formula I would be higher over this temperature range as a function of the material of formula I content and the partial pressure exerted by the material of formula I, e.g., diethylene glycol and also of the products formed.

Because the water is being utilized at near critical or at super critical conditions, it is not necessary to include any catalytic components in the reactant mixture. Thus, in a preferred embodiment the reaction mixtures contain only the material of formula I and water.

It is possible, however, to utilize a trace quantity of a solid, liquid or gaseous acidic material such as phosphoric acid, sulfuric acid, MCM-22, hydrochloric acid, HF, acetic acid, zeolite, etc., preferably phosphoric acid.

The amount of added acid used, if any is present at all, is minimal. Thus, the amount of added acid ranges from about zero to about 5 wt % of the total mixture, preferably about zero to about 1.0 wt % of the total mixture, more preferably about zero to about 0.05 wt % of the total mixture, most preferably about zero to 0.01 wt %.

The process of this invention may be carried out batchwise or in the continuous mode using conventional pressure equipment. Examples of such equipment include a laboratory bomb, a high pressure autoclave, a stirred tank reactor or continuous flow through tubes, each equipped with heating means and sealing means capable of achieving and maintaining the required temperatures and pressures over the time period necessary for reaction to occur to a desirable level of conversion.

### EXAMPLES

Synthesis of dioxane from diethylene glycol.

All of the reactions were conducted in Swagelock® stainless steel mini-reactors, each mini-reactor consisted of a cap and plug and were either of 12, 6 or 3 ml volume. The tared mini-reactors were charged with water and DEG to a total charge weight of about 1-5.25 g as indicated in the following table and optional trace of acid under a nitrogen atmosphere and immediately sealed and re-weighed. The sealed reactors were heated in a preheated silicone oil bath, to either 250° C., 300° C., or 350° C. The hot reactors were removed from the hot oil bath after heating for a given time period and submerged in room temperature water for rapid cooling to room temperature. The cooled mini-reactors were rinsed with toluene to wash any residual silicone oil off the mini-reactor. The mini-reactors were re-weighed to establish that no weight loss had occurred before being opened in the hood. The liquid phase was analyzed by gas chromatography using a Hewlett-Packard 6890 gc containing a 30-meter non-polar HP-1 (cross-linked methylsiloxane) column. Table 1 provides yields of dioxane product using the percentages from the gc analyses:

TABLE 1

| Run | | DEG + | | | | DEG | Dioxane |
|---|---|---|---|---|---|---|---|
| # | H₂O:DEG | H₂O | | Temp. | Time | Conv. | Yield |
| 23686 | (wt ratio) | (g) | Other Components | (° C.) | (min) | (%) | (%) |
| 33-1 | 5 to 1 | 3.99 | None | 250 | 30 | 0.0 | 0 |
| 33-2 | 5 to 1 | 3.93 | None | 250 | 60 | 0.6 | 0 |
| 33-3 | 5 to 1 | 4.00 | None | 250 | 90 | 0.5 | 0 |
| 33-4 | 5 to 1 | 5.23 | None | 300 | 20 | 4.9 | 25.8 |
| 62-1 | 5 to 1 | 1.0 | None | 250 | 360 | 2.4 | 17.3 |
| 62-2 | 5 to 1 | 1.0 | None | 300 | 60 | 7.1 | 23.8 |
| 62-3 | 5 to 1 | 1.0 | None | 300 | 360 | 45.5 | 20.1 |
| 62-4 | 5 to 1 | 1.0 | None | 350 | 20 | 8.1 | 20.5 |
| 62-5 | 5 to 1 | 1.0 | None | 350 | 60 | 23.2 | 22.0 |
| 33-5 | 5 to 1 | 1.96 | 0.0356 g 1 M H₂SO₄ (1 | 300 | 20 | 59.5 | 30.0 |
| | | | drop)* | | | | |
| 33-6 | 5 to 1 | 2.02 | 0.0356 g 1 M H₂SO₄ (1 | 300 | 60 | 84.1 | 22.0 |
| | | | drop) | | | | |
| 36-1 | 5 to 1 | 1.89 | 0.0339 g H⁺BETA, PG | 300 | 60 | 40.1 | 34.2 |
| | | | Corporation | | | | |
| 36-2 | 5 to 1 | 2.00 | 0.0096 g MCM-22 | 300 | 60 | 65.5 | 46.7 |
| 36-3 | 5 to 1 | 2.05 | 0.0372 g 0.5 M H₂SO₄ (1 | 300 | 60 | 41.3 | 17.4 |
| | | | drop) | | | | |
| 36-4 | 5 to 1 | 2.00 | 0.0372 g 0.5 M H₂SO₄ (1 | 300 | 60 | 68.7 | 21.2 |
| | | | drop) | | | | |
| 38-1 | 5 to 1 | 1.93 | 0.0372 g 1 M H₃PO₄ (1 | 300 | 60 | 70.7 | 24.7 |
| | | | drop) | | | | |
| 38-2 | 5 to 1 | 1.90 | 0.0372 g 1 M H₃PO₄ (1 | 300 | 60 | 59.3 | 21.0 |
| | | | drop) | | | | |
| 38-3 | 5 to 1 | 1.50 | 0.0696 g 1 M H₃PO₄ (2 | 300 | 60 | 83.2 | 24.8 |
| | | | drops) | | | | |
| 38-4 | 5 to 1 | 2.00 | 0.0696 g 1 M H₃PO₄ (2 | 300 | 60 | 69.1 | 23.4 |
| | | | drops) | | | | |
| 38-5 | 5 to 1 | 2.00 | 0.0372 g 1 M H₃PO₄ (1 | 300 | 60 | 68.7 | 22.7 |
| | | | drop) | | | | |
| 38-6 | 5 to 1 | 2.00 | 0.0372 g 1 M H₃PO₄ (1 | 300 | 120 | 79.5 | 27.0 |
| | | | drop) | | | | |
| 38-7 | 5 to 1 | 1.95 | 0.0372 g 1 M H₃PO₄ (1 | 300 | 120 | 96.2 | 34.0 |
| | | | drop) | | | | |
| 38-8 | 5 to 1 | 1.43 | 0.0372 g 1 M H₃PO₄ (1 | 300 | 180 | 94.9 | 35.6 |
| | | | drop) | | | | |
| 38-9 | 5 to 1 | 0.98 | 0.0372 g 1 M H₃PO₄ (1 | 300 | 180 | 95.7 | 32.0 |
| | | | drop) | | | | |
| 38-10 | 1 to 1 | 2.00 | 0.0372 g 1 M H₃PO₄ (1 | 300 | 120 | 77.1 | 42.1 |
| | | | drop) | | | | |
| 40-1 | 0.1 to 1 | 1.23 | 0.0372 g 1 M H₃PO₄ (1 | 300 | 180 | 51.5 | 34.6 |
| | | | drop) | | | | |
| 40-2 | 0.1 to 1 | 1.23 | 0.0035 g MCM-22 | 300 | 180 | 70.0 | 56.8 |
| 40-3 | 0.1 to 1 | 1.23 | None | 300 | 180 | 10.6 | 19.0 |
| 40-4 | 0.1 to 1 | 1.23 | None | 300 | 360 | 11.9 | 19.8 |
| 44-1 | 0.13 to 1 | 1.21 | 0.0063 g MCM-22 | 300 | 120 | 79.2 | 67.3 |
| 44-2 | 0.11 to 1 | 1.20 | 0.0056 g MCM-22 | 300 | 240 | 90.9 | 70.9 |
| 44-3 | 0.12 to 1 | 1.21 | 0.0084 g MCM-22 | 300 | 360 | 91.2 | 68.9 |
| 44-4 | Only | 1.11 | 0.0071 g MCM-22 | 300 | 120 | 80.2 | 58.1 |
| | DEG** | | | | | | |
| 44-5 | Only | 1.02 | 0.0063 g MCM-22 | 300 | 240 | 85.3 | 61.6 |
| | DEG** | | | | | | |
| 44-6 | Only | 1.04 | 0.0075 g MCM-22 | 300 | 360 | 93.1 | 64.4 |
| | DEG** | | | | | | |

*1 drop = 0.05 ml
**DEG contained 117 ppm water (˜0.00012 g H₂O)/g DEG). The MCM-22 sample used contained 4.86% water absorbed from the atmosphere (˜0.0003 g H₂O)/g MCM-22). Thus, examples 44-4/5/6 contained about 0.0004 g H₂O just from the DEG and the MCM-22 catalyst, a water to DEG mole ratio of about 0.002:1.

The results in Table 1 illustrate that DEG is converted to p-dioxane in superheated water. However, addition of trace amounts of an acid catalyst enhances the conversion to over 70% yield in 4 hr at 300° C.

Synthesis of morpholine from 2,2′ oxybis(ethylamine)

0.5 g (0.0028 mole) of 2,2′ oxybis(ethylamine) dihydrochloride was dissolved in 2.5 g of distilled water. Sodium bicarbonate (0.475 g, 0.0057 mole) was added to neutralize the HCl resulting in the liberation of the diamine starting material.

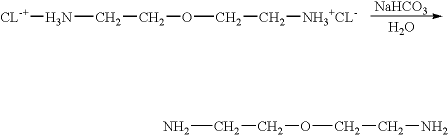

An additional 2.5 g of distilled water was added to dissolve the solids. 1.0 g of the resulting clear light yellow liquid (10:1 water diamine) was added to a Swagelock cap and plug reactor and the contents were heated for 1 hour at 300° C. The resulting solution was then sampled and analyzed by gc and gc/ms to confirm the morpholine product. Complete conversion (100%) of the diamine yielded 41.8% morpholine.

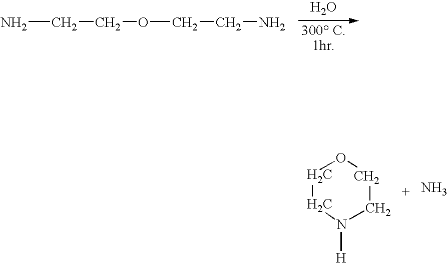

1. A process for the direct synthesis of di-heteroatom containing cyclic organic compounds by forming an aqueous mixture of water and a material of the formula 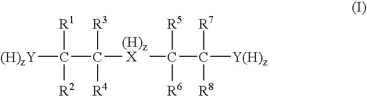
wherein X and Y are heteroatoms which may be the same or different and wherein X is O, S or N, and both Y's are the same and are O, S or N, and R¹, R², R³, R⁴, R⁵, R⁶, R⁷ and R⁸ are the same or different and are hydrogen, C₁ to C₁₀ alkyl radicals, C₁ to C₁₀ aryl radicals or C₁ to C₁₀ alkyl aryl radicals, and z is zero when X and Y are O or S, and z is 1 when X and Y are N, the water and material of formula I being used on a mole basis in the ratio of about 10:1 to about 0.001:1, the mixture being heated under autogeneous pressure at a temperature within the range of from about 200° C. to about 450° C. for a time sufficient to cause dehydration, dehydrosulfurization or deamination and ring closure of the material of formula I.

2. The process of claim 1 wherein X is O.

3. The process of claim 1 wherein Y is O.

4. The process of claim 1 wherein X is S and Y is O.

5. The process of claim 1 wherein X is S and Y is S.

6. The process of claim 1 wherein X is N and Y is N.

7. The process of claim 1 wherein X is N and Y is N.

8. The process of claim 1 wherein X is S and Y is N.

9. The process of claim 1 wherein X is O and Y is O.

10. The process of claim 1 wherein R¹, R², R³, R⁴, R⁵, R⁶ and R⁷ are H.

11. The process of claim 1 in which the aqueous mixture further comprises an acid in an amount in the range of up to about 5 wt % of the total mixture.

12. The process of claim 11 wherein the acid is present in an amount up to about 1.0 wt % of the total mixture.

13. The process of claim 12 wherein the acid is present in an amount up to about 0.05 wt % of the total mixture.

14. The process of claim 1 wherein the mole ratio of water to material of formula I is from about 5:1 to about 0.001:1.

15. The process of claim 10 in which the aqueous mixture further comprises an acid in an amount in the range of up to about 5 wt % of the total mixture.

16. The process of claim 15 wherein the acid is present in an amount up to about 1.0 wt % of the total mixture.

17. The process of claim 16 wherein the acid is present in an amount up to about 0.05 wt % of the total mixture.

18. The process of claim 15 wherein the mole ratio of water to material of formula I is from about 5:1 to about 0.001:1.

19. The process of claim 15 in which X is O and Y is O.

20. The process of claim 17 and 18 in which X is O and Y is O.
